(19) United States
(12) Patent Application Publication
(10) Pub. No.: US 2023/0270404 A1
(43) Pub. Date: Aug. 31, 2023

(54) CAROTID ARTERY ULTRASONIC EXAMINATION REPORT GENERATION SYSTEM BASED ON MULTI-MODAL INFORMATION

(71) Applicant: SHANDONG UNIVERSITY, Qingdao, Shandong (CN)

(72) Inventors: Zhi LIU, Qingdao (CN); Yankun CAO, Qingdao (CN)

(73) Assignee: SHANDONG UNIVERSITY, Qingdao, Shandong (CN)

(21) Appl. No.: 18/024,320

(22) PCT Filed: Sep. 5, 2022
(86) PCT No.: PCT/CN2022/117080

§ 371 Date: Mar. 2, 2023

(30) Foreign Application Priority Data
Oct. 13, 2021 (CN) 202111192140X

(51) Int. Cl. A61B 8/08; A61B 8/06

(52) U.S. Cl. CPC A61B 8/0891; A61B 8/06; A61B 8/488; A61B 8/5223

(57) ABSTRACT

A carotid artery ultrasonic examination report generation system which includes an ultrasonic device which acquires multi-modal information of a to-be-examined carotid artery; the processor is connected with the ultrasonic device, and includes: a plaque type recognition module that inputs multi-modal information into a plaque type recognition model to obtain the carotid artery's plaque type; an image division module inputs an ultrasonic image into different division models according to plaque type to obtain a divided image set; an abnormity detection module calculates image parameter set based on divided image set and input image parameter set into a carotid artery abnormity detection model to obtain a result indicating whether the carotid artery is abnormal; and a carotid artery ultrasonic examination report generation module generates a report based on the image parameter set, plaque type, blood flow spectrum pattern, blood flow kinetic parameters and result indicates whether the carotid artery is abnormal.

### TECHNICAL FIELD

The present disclosure belongs to the technical field of carotid artery ultrasonic examination report generation, and particularly relates to a carotid artery ultrasonic examination report generation system based on multi-modal information.

### BACKGROUND

The statements in this section merely provide background related to the present disclosure and may not necessarily constitute the prior art.

Carotid artery stenosis may lead to a variety of brain diseases and is diagnosed through imageological examination on carotid artery at present. Currently, there are many problems at the community level, such as the shortage of high-level doctors, the imbalance of medical resources between urban and rural areas, and the limitation of device imaging accuracy. If imageological images are judged only by doctors with naked eyes, the workload will be great, and misdiagnosis may occur due to the subjectivity of the doctors.

In order to solve the above problems, there is an urgent need for a carotid artery ultrasonic examination report generation system for early screening and diagnosis assistance to greatly reduce the workload of doctors and improve the diagnosis efficiency.

### SUMMARY

In order to solve the above problems, the present disclosure provides a carotid artery ultrasonic examination report generation system based on multi-modal information. By acquiring and processing multi-modal information of carotid artery, vascular diseases are automatically recognized, and ultrasonic examination reports are output to assist diagnosis by a doctor, thus reducing the burden of the doctor and improving the diagnosis efficiency.

According to some embodiments, the present disclosure adopts the following technical solution:
a carotid artery ultrasonic examination report generation system based on multi-modal information includes:
an ultrasonic device, configured to acquire multi-modal information of a to-be-examined carotid artery, the multi-modal information including an ultrasonic image, a Doppler color blood flow image, a blood flow spectrum pattern and blood flow kinetic parameters, and
a processor, connected with the ultrasonic device and including:
a plaque type recognition module, configured to input the multi-modal information into a plaque type recognition model to obtain a plaque type of the to-be-examined carotid artery;
an image division module, configured to input the ultrasonic image into different division models according to the plaque type to obtain a divided image set;
an abnormity detection module, configured to calculate an image parameter set based on the divided image set and input the image parameter set into a carotid artery abnormity detection model to obtain a result indicating whether the carotid artery is abnormal or not; and
a carotid artery ultrasonic examination report generation module, configured to generate a carotid artery ultrasonic examination report based on the image parameter set, the plaque type, the blood flow spectrum pattern, the blood flow kinetic parameters and the result indicating whether the carotid artery is abnormal or not at a set ultrasonic examination report format.

Further, the image division module includes:
a plaque type determination unit, configured to determine whether the plaque type belongs to no plaque or not, if YES, input the ultrasonic image into a tunica intima and externa division unit, and if NO, input the ultrasonic image into the tunica intima and externa division unit and a plaque division unit;
the tunica intima and externa division unit, configured to input the ultrasonic image into a tunica intima and externa division model to obtain a tunica intima and externa divided image and add the tunica intima and externa divided image into the divided image set; and
the plaque division unit, configured to input the ultrasonic image into a plaque division model to obtain a plaque divided image and add the plaque divided image into the divided image set.

Further, the abnormity detection module includes:
a divided image determination unit, configured to determine whether the plaque divided image is in the divided image set or not, if YES, input the plaque divided image into a plaque area calculation unit and a plaque volume calculation unit and input the tunica intima and externa divided image into a tunica intima and externa width calculation unit at the same time; and if NO, input the tunica intima and externa divided image into the tunica intima and externa width calculation unit.

Further, the tunica intima and externa width calculation unit is configured to: calculate a length of a single pixel based on the length and the pixel number of the ultrasonic image;
count the tunica intima and externa pixel number in each frame of the tunica intima and externa divided image;
calculate the tunica intima and externa width of each frame of the tunica intima and externa divided image based on the length of the single pixel and the tunica intima and externa pixel number; and
select the greatest tunica intima and externa width as a final tunica intima and externa width to be added into the image parameter set, and transmit a frame of tunica intima and externa divided image with the greatest tunica intima and externa width to a carotid artery stenosis rate calculation unit.

Further, the carotid artery stenosis rate calculation unit is configured to:
receive the frame of tunica intima and externa divided image with the greatest tunica intima and externa width, calculate a distance among all tunica intima pixels in a line for each line in an image matrix of the frame of image, and select the greatest distance as a tunica intima diameter; and
select the smallest diameter and the greatest diameter in all the tunica intima diameters, take a ratio of the smallest diameter to the greatest diameter as a carotid artery stenosis rate, and add the carotid artery stenosis rate to the image parameter set.

Further, the plaque area calculation unit is configured to: calculate a length of a single pixel based on the length and the pixel number of the ultrasonic image;
count the pixel number of the plaque in each frame of the plaque divided image;
calculate a plaque area of each frame of the plaque divided image based on the length of the single pixel and the pixel number of the plaque; and
select the greatest plaque area as a final plaque area to be added to the image parameter set, and transmit the frame of plaque divided image with the greatest plaque area to the plaque volume calculation unit.

Further, the plaque volume calculation unit is configured to:
count a frame number of the ultrasonic image with the plaque in the plaque divided image;
receive the frame of plaque divided image with the greatest plaque area, and draw a smallest rectangle capable of completely covering a plaque region with a plaque division boundary as a standard in the frame of image, where a length of the rectangle is a plaque major axis diameter, and a width of the rectangle is a plaque minor axis diameter; and
calculate a plaque volume based on the plaque major axis diameter, the plaque minor axis diameter and the frame number of the ultrasonic image with the plaque, and add the plaque volume to the image parameter set.

Further, the processor further includes a user feedback module, which is configured to obtain user feedback information and update the plaque type recognition model, the tunica intima and externa division model or the plaque division model based on the user feedback information.

Further, the user feedback information is one or more of no modification, a final conclusion modification result, a tunica intima and externa divided image modification result and a plaque divided image modification result;
the final conclusion modification result is used as feedback to be input into the plaque type recognition module, a modified plaque type and corresponding carotid artery multi-modal information are added into a training set to retrain the plaque type recognition model to update parameters of the plaque type recognition model;
the tunica intima and externa divided image modification result is used as feedback to be input into the tunica intima and externa division unit to update parameters of the tunica intima and externa division model; and
the plaque divided image modification result is used as feedback to be input into the plaque division unit to update parameters of the plaque division model.

Further, a display device is further included, is connected with the processor, and is configured to display the tunica intima and externa divided image, the plaque divided image and the carotid artery ultrasonic examination report.

Compared with the prior art, the present disclosure has the following beneficial effects:

The present disclosure provides the carotid artery ultrasonic examination report generation system based on multi-modal information. By acquiring and processing the carotid artery multi-modal information, vascular diseases can be automatically recognized, and ultrasonic examination reports are output to assist diagnosis of the doctor, thus improving the report generation accuracy, reducing the burden of doctors and improving the diagnosis efficiency at the same time.

The present disclosure provides the carotid artery ultrasonic examination report generation system based on multi-modal information, the plaque type recognition model, the tunica intima and externa division model or the plaque division model is updated based on the user feedback information, the problems of lack of carotid artery labels and small sample are solved, and the model accuracy is further improved.

### BRIEF DESCRIPTION OF THE DRAWINGS

The accompanying drawings which constitute a part of the present disclosure are used for providing a further understanding of the present disclosure. The schematic embodiments and their descriptions are used for explaining the present disclosure and do not constitute an undue limitation on the present disclosure.

FIG. 1 is a frame diagram of a carotid artery ultrasonic examination report generation system based on multi-modal information according to Embodiment 1 of the present disclosure.

FIG. 2 is a frame diagram of a plaque type recognition model according to Embodiment 1 of the present disclosure.

### DETAILED DESCRIPTION

The present disclosure will be further illustrated in detail in conjunction with accompanying drawings and embodiments hereafter.

It should be pointed out that the following detailed descriptions are all exemplary and are intended to provide further explanation on the present disclosure. Unless otherwise specified, all technical and scientific terms used herein have the same meaning as commonly understood by ordinary skill in the art to which the present disclosure belongs.

It should be noted that the terms used herein are merely for the purpose of describing specific embodiments and are not intended to limit exemplary embodiments in accordance with the present disclosure. As used herein, unless it is clearly indicated otherwise in the context, the singular forms are also intended to include the plural forms, additionally, it is to be understood that the terms “include” and/or “comprise”, when used in this specification, specify the presence of features, steps, operations, elements, components, and/or combinations thereof.

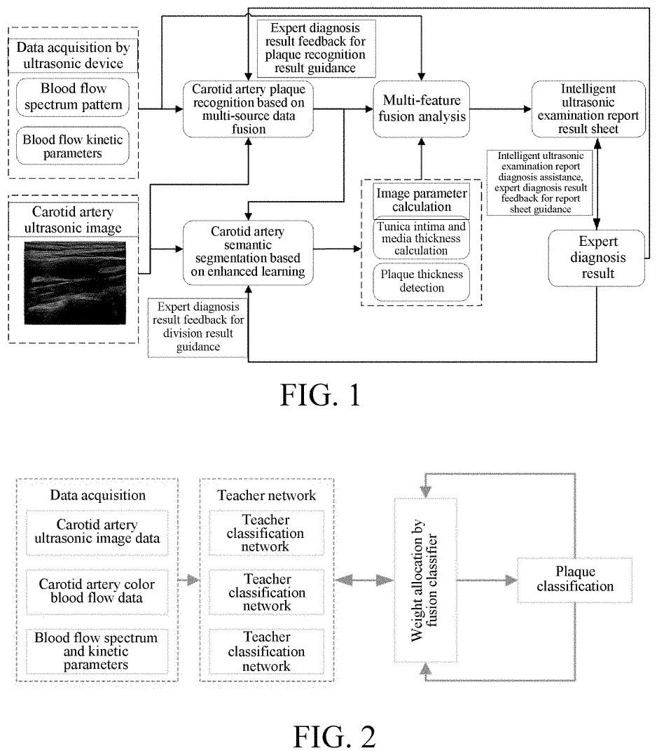

As shown in FIG. 1, a carotid artery ultrasonic examination report generation system based on multi-modal information according to the embodiment automatically recognizes vascular diseases and outputs ultrasonic examination reports by acquiring and processing carotid artery multi-modal information, and includes an ultrasonic device, a processor and a display device.

The ultrasonic device is configured to acquire multi-modal information of a to-be-examined carotid artery, and the multi-modal information includes an ultrasonic image, a Doppler color blood flow image, a blood flow spectrum pattern and blood flow kinetic parameters.

As an implementation, the carotid artery multi-modal information includes the ultrasonic image, the Doppler color blood flow image and Doppler spectrum ultrasonic information of a carotid artery of a patient.

As an implementation, the ultrasonic image, the Doppler color blood flow image and the Doppler spectrum ultrasonic information of the carotid artery are acquired by the ultrasonic device. The ultrasonic device includes but is not limited to an ultrasonic acquisition instrument, a handheld ultrasonic device, a 5G remote ultrasonic acquisition device, etc. Different from a host and probe product form of a traditional ultrasonic device, a host is reduced to a very small circuit board built in a probe, achieving an effect that one probe equals to a B-mode ultrasonic device capable of realizing display only with the help of a carry-on mobile phone or tablet computer installed with ultrasonic APP software, and an image is transmitted to the mobile phone/tablet computer through probe built-in wifi. The carotid artery multi-modal information is transmitted to the processor through the probe built-in wifi.

The ultrasonic image has a plurality of frames, can be used for obtaining two-dimensional carotid artery image information, and can be used for subsequent carotid artery tunica intima and externa division and plaque division to obtain a carotid artery tunica intima and externa width, a stenosis degree, and a plaque length, width and area. Through the Doppler color blood flow image, an intraluminal blood flow filling condition can be obtained, and the carotid artery blood flow change is displayed with colors. Through the Doppler spectrum ultrasonic information, the blood flow spectrum pattern and the carotid artery blood flow kinetic parameters can be obtained, and the carotid artery blood flow kinetic parameters include systole double peaks, diastole duration, forward blood flow movement, etc.

The processor is connected with the ultrasonic device, and includes a plaque type recognition module, an image division module, an abnormity detection module, a carotid artery ultrasonic examination report generation module and a user feedback module.

The plaque type recognition module is configured to input the multi-modal information of the to-be-examined carotid artery into a trained plaque type recognition model to obtain a plaque type of the to-be-examined carotid artery.

The plaque type includes three types: no plaque, hard plaque and soft plaque. The plaque type recognition model is built based on a knowledge distillation network, and the plaque type recognition model is obtained by inputting a training set into the plaque type recognition model based on the knowledge distillation network. The training set includes multi-modal information of a plurality of carotid arteries and labeled plaque types thereof. The knowledge distillation network includes a teacher network and a student network. The teacher network has a label for weight learning, and then transmits parameters to the student network, and the student network has no label.

The plaque type recognition model uses a multi-modal data feature extraction and fusion method based on the knowledge distillation network to fuse the multi-modal data acquired in step 1 and performs plaque type detection, as shown in FIG. 2. By aiming at the specificity of the carotid artery data, a multi-modal data feature extraction and fusion method based on a knowledge distillation model is provided for plaque type detection, and includes the following specific steps: a carotid artery ultrasonic image, a carotid artery Doppler color blood flow image, a blood flow spectrum pattern, blood flow kinetic parameters are transmitted into the teacher network for learning, then, different weights are given to each network through a fusion classifier, a result is fed back to each sub network according to a label, and a detection result is finally output.

The image division module is configured to input the ultrasonic image into different division models according to the plaque type to obtain a divided image set. The division model includes a plaque division model and a tunica intima and externa division model. The image division module includes a plaque type determination unit, a tunica intima and externa division unit and a plaque division unit.

The plaque type determination unit is configured to determine whether the plaque type belongs to no plaque or not, if YES, input the ultrasonic image into the tunica intima and externa division unit, and if NO, input the ultrasonic image into the tunica intima and externa division unit and the plaque division unit.

The tunica intima and externa division unit is configured to input the ultrasonic image into the tunica intima and externa division model to obtain a tunica intima and externa divided image and add the tunica intima and externa divided image into the divided image set.

The plaque division unit is configured to input the ultrasonic image into the plaque division model to obtain a plaque divided image and add the plaque divided image into the divided image set.

The tunica intima and externa division model is obtained by training a tunica intima and externa division model based on a semantic segmentation network by a small amount of sample images marked with tunica intima and externa regions. The plaque division model is obtained by training a plaque division model based on a semantic segmentation network by a small amount of sample images marked with plaque regions. The trained tunica intima and externa division model and plaque division model are used for respectively performing pixel level division on the carotid artery tunica intima and externa and the plaque of the patient in the ultrasonic image.

As an implementation, the semantic segmentation network includes but is not limited to mainstream semantic segmentation networks such as FCN, Deeplab, Unet, etc.

As an implementation, the semantic segmentation network performs training in a semi-supervised enhanced learning mode to obtain the tunica intima and externa division model or the plaque division model, and specifically includes the following steps: firstly, a carotid artery ultrasonic image is acquired, a small amount of data sets are made and marked, and the data sets are trained in an enhanced learning mode. A major enhanced learning method is as follows: the carotid artery tunica intima and externa or plaque positions are positioned according to labels, then, the position information is input into the enhanced network, and the network finds a best matching position according to the labels, so that the tunica intima and externa and plaque division effect is achieved. The semi-supervised mode is embodied in the following aspects: when the trained network is tested, a test result is fed back to an expert using the system at any time, the expert judges the output effect of the network, if it is approved by the expert, the result is output as a final result, if it is not approved by the expert, the result is continuously returned to the network to be trained until the result is approved by the expert; and finally, the trained semantic segmentation network is output for dividing the carotid artery tunica intima and externa or plaque in the ultrasonic image.

The abnormity detection module is configured to calculate an image parameter set based on the divided image set and input the image parameter set into a carotid artery abnormity detection model to obtain a result indicating whether the carotid artery is abnormal or not. The abnormity detection module includes a divided image determination unit, a plaque area calculation unit, a plaque volume calculation unit, a tunica intima and externa width calculation unit, a carotid artery stenosis rate calculation unit and a carotid artery abnormity determination unit.

The divided image determination unit is configured to determine whether the plaque divided image is in the divided image set or not, if YES, input the plaque divided image into the plaque area calculation unit and the plaque volume calculation unit and input the tunica intima and externa divided image into the tunica intima and externa width calculation unit at the same time; and if NO, input the tunica intima and externa divided image into the tunica intima and externa width calculation unit.

After the tunica intima and externa division and plaque division, a tunica intima and externa division result picture and a plaque division picture may be obtained, the tunica intima and externa width and the plaque size and area may be obtained through calculation according to the length relationship represented by each pixel in a practical ultrasonic image, and the plaque volume may be obtained through calculation according to the plaque area of a plurality of frames of images. Specifically, the pixels of the divided region (plaque or tunica intima and externa) are obtained through calculation by using the obtained tunica intima and externa divided image and plaque divided image, and the pixel measurement is converted into distance measurement according to a correspondence of the pixels and medical measurement, so that the tunica intima and externa width information may be obtained through calculation. For the plaque area calculation, the length and the width can be obtained through calculation according to the pixel correspondence, so that the area calculation can be performed.

The tunica intima and externa width calculation unit is configured to: calculate a length of a single pixel based on the length and the pixel number of the ultrasonic image, specifically, each ultrasonic acquisition device is provided with the length of the carotid artery ultrasonic image, supposed that the length of the ultrasonic image displayed by the device for acquiring this section of the carotid artery ultrasound is 1, an output ultrasonic image matrix is n*n, namely the pixel number is n*n, and the length of the single pixel is d=l/n; count the tunica intima and externa divided pixel number ml in each frame of tunica intima and externa divided image; calculate the tunica intima and externa width h=m1*d of each frame of the tunica intima and externa divided image based on the length of the single pixel and the tunica intima and externa pixel number; select the greatest tunica intima and externa width as a final tunica intima and externa width; and add the final tunica intima and externa width into the image parameter set, and transmit a frame of tunica intima and externa divided image with the greatest tunica intima and externa width to the carotid artery stenosis rate calculation unit.

The plaque area calculation unit is configured to: calculate a length of a single pixel based on the length and the pixel number of the ultrasonic image; count the pixel numberm2 of the plaque in each frame of the plaque divided image; calculate the plaque area s=m2*d of each frame of the plaque divided image based on the length of the single pixel and the pixel number of the plaque; select the greatest plaque area as a final plaque area; and add the final plaque area to the image parameter set, and transmit a frame of plaque divided image with the greatest plaque area to the plaque volume calculation unit.

The carotid artery stenosis rate calculation unit is configured to: receive the frame of tunica intima and externa divided image with the greatest tunica intima and externa width, calculate a distance among all tunica intima pixels in a line for each line in an image matrix of the frame of tunica intima and externa divided image, and select the greatest distance in each line as a tunica intima diameter of the line; and select the smallest diameter and the greatest diameter in all the tunica intima diameters, and take a ratio of the smallest diameter to the greatest diameter as a carotid artery diameter stenosis rate. In other words, from the first pixel of the image, at the moment of meeting the first tunica intima pixel, a perpendicular line is drawn downwards from the first tunica intima pixel until the perpendicular line intersects with another tunica intima line, this line section is regarded as a carotid artery diameter (i.e., the tunica intima diameter), the first line of the image matrix is traversed to obtain all the tunica intima diameters, the ratio of the smallest diameter to the greatest diameter is taken as the carotid artery diameter stenosis rate, and the carotid artery stenosis rate is added into the image parameter set.

The plaque volume calculation unit is configured to: count a frame number f of the ultrasonic image with the plaque in the plaque divided image since the volume of the plaque may be approximately an ellipsoid while the height represented by each frame is h (h may be obtained during device acquisition); receive the frame of plaque divided image with the greatest plaque area, and draw a smallest rectangle capable of completely covering a plaque region with a plaque division boundary as a standard in the frame of image, a length of the rectangle is a plaque major axis diameter, and a width of the rectangle is a plaque minor axis diameter; and calculate a plaque volume 4πabc/3 based on the plaque major axis diameter, the plaque minor axis diameter and the frame number of the ultrasonic image with the plaque, a is a half of the frame number f*h, b is a half of the plaque major axis diameter, and c is a half of the plaque minor axis diameter; and add the plaque volume to the image parameter set.

The carotid artery abnormity determination unit performs multi-feature fusion analysis, performs integral judgment based on the blood flow spectrum pattern, the blood flow kinetic parameters, the tunica intima and externa width, the carotid artery stenosis rate, and the plaque type, area and volume, and performs detection to obtain a result indicating whether the carotid artery is abnormal or not.

The carotid artery abnormity determination unit is configured to input the blood flow spectrum pattern, the blood flow kinetic parameters, the tunica intima and externa width, the carotid artery stenosis rate, and the plaque type, area and volume into the trained carotid artery abnormity detection model to obtain the result indicating whether the carotid artery is abnormal or not. The carotid artery abnormity detection model is obtained by training a carotid artery abnormity detection model based on a neural network by a training set, and the training set includes the blood flow kinetic parameters, the tunica intima and externa width, the carotid artery stenosis rate, the plaque type, area and volume, and the corresponding marked result indicating whether the carotid artery is abnormal or not.

The carotid artery abnormity determination unit integrates each piece of information for diagnosis to obtain the result indicating whether the carotid artery is abnormal or not, and outputs the result on the ultrasonic examination report, and the result indicates whether the carotid artery is abnormal or not.

The carotid artery ultrasonic examination report generation module is configured to generate a carotid artery ultrasonic examination report based on the image parameter set, the plaque type, the blood flow spectrum pattern, the blood flow kinetic parameters and the result indicating whether the carotid artery is abnormal or not at a set ultrasonic examination report format.

As this implementation, the ultrasonic examination report further includes ultrasonic hints, and the ultrasonic hints are obtained by a set determination rule according to integral information.

The user feedback module is configured to obtain user feedback information, update the ultrasonic examination report based on the user feedback information and update the plaque type recognition model, the tunica intima and externa division model or the plaque division model based on the user feedback information. The user feedback information is one or more of no modification, a final conclusion modification result, a tunica intima and externa divided image modification result and a plaque divided image modification result.

The plaque divided image, the tunica intima and externa divided image and the ultrasonic examination report are fed back to a user together. In order to effectively reduce the error rate and improve the robustness instruction of an artificial intelligent system for the user, the expert can modify the ultrasonic examination report information through directly modifying the final conclusion, and can modify the report information through parameter division guidance by modifying the plaque divided image and the tunica intima and externa divided image. The final conclusion modification result (user modified plaque type) is used as feedback to be input into the plaque type recognition module, the modified plaque type and the corresponding carotid artery multi-modal information are added into the training set to retrain the plaque type recognition model, so as to update parameters of the plaque type recognition model. The tunica intima and externa divided image modification result or the plaque divided image modification result is used as feedback to be input into the tunica intima and externa division unit or the plaque division unit, so as to update parameters of the tunica intima and externa division model or the plaque division model. The accuracy of the training model is further improved.

The display device is connected with the processor, and is configured to display the tunica intima and externa divided image, the plaque divided image and the carotid artery ultrasonic examination report.

An ultrasonic examination report result sheet is obtained in combination with the plaque thickness information obtained through the tunica intima and media division result of the carotid artery image according to the present disclosure, and an intelligent diagnosis assistance effect is achieved. The expert can obtain the self diagnosis conclusion according to the intelligent ultrasonic examination report sheet. The conclusion can be fed back to guide the result of the report sheet, and can be fed back to guide the plaque detection result and the tunica intima and media division result.

The present disclosure performs carotid artery screening in a multi-modal semi-supervised mode, the report generation accuracy is improved, at the same time, the burden of the doctor is reduced, and the diagnosis efficiency is improved.

Finally, it should be noted that the above embodiments are only used for illustrating the technical solution of the present disclosure but not intended to limit the present disclosure. Although the present disclosure has been described in detail with reference to the above embodiments, it should be understood by those skilled in the art that modifications or equivalent substitutions may be made to the embodiments of the present disclosure without departing from the spirit and scope of the present disclosure, and all the modifications or equivalent substitutions shall fall within the protection scope of claims of the present disclosure.

What is claimed is:

1. A carotid artery ultrasonic examination report generation system based on multi-modal information, comprising:
an ultrasonic device, configured to acquire multi-modal information of a to-be-examined carotid artery, the multi-modal information comprising an ultrasonic image, a Doppler color blood flow image, a blood flow spectrum pattern and blood flow kinetic parameters, and
a processor, connected with the ultrasonic device and comprising:
a plaque type recognition module, configured to input the multi-modal information into a plaque type recognition model to obtain a plaque type of the to-be-examined carotid artery;
an image division module, configured to input the ultrasonic image into different division models according to the plaque type to obtain a divided image set;
an abnormity detection module, configured to calculate an image parameter set based on the divided image set and input the image parameter set into a carotid artery abnormity detection model to obtain a result indicating whether the carotid artery is abnormal or not; and
a carotid artery ultrasonic examination report generation module, configured to generate a carotid artery ultrasonic examination report based on the image parameter set, the plaque type, the blood flow spectrum pattern, the blood flow kinetic parameters and the result indicating whether the carotid artery is abnormal or not at a set ultrasonic examination report format;
wherein the plaque type recognition model utilizes a multi-modal data feature extraction and fusion method based on a knowledge distillation network, the multi-modal data feature extraction and fusion method based on the knowledge distillation network is used for plaque type detection, and comprises the following specific steps: transmitting the carotid artery ultrasonic image, the carotid artery Doppler color blood flow image, the blood flow spectrum pattern and the blood flow kinetic parameters into a teacher network for learning, then, giving different weights to each network through a fusion classifier, feeding a result to each sub network according to a label, and finally outputting a detection result.

2. The carotid artery ultrasonic examination report generation system based on multi-modal information according to claim 1, wherein the image division module comprises:
a plaque type determination unit, configured to determine whether the plaque type belongs to no plaque or not, if YES, input the ultrasonic image into a tunica intima and externa division unit, and if NO, input the ultrasonic image into the tunica intima and externa division unit and a plaque division unit;
the tunica intima and externa division unit, configured to input the ultrasonic image into a tunica intima and externa division model to obtain a tunica intima and externa divided image and add the tunica intima and externa divided image into the divided image set; and
the plaque division unit, configured to input the ultrasonic image into a plaque division model to obtain a plaque divided image and add the plaque divided image into the divided image set.

3. The carotid artery ultrasonic examination report generation system based on multi-modal information according to claim 2, wherein the abnormity detection module comprises:
a divided image determination unit, configured to determine whether the plaque divided image is in the divided image set or not, if YES, input the plaque divided image into a plaque area calculation unit and a plaque volume calculation unit and input the tunica intima and externa divided image into a tunica intima and externa width calculation unit at the same time;
and if NO, input the tunica intima and externa divided image into the tunica intima and externa width calculation unit.

4. The carotid artery ultrasonic examination report generation system based on multi-modal information according to claim 3, wherein the tunica intima and externa width calculation unit is configured to:
calculate a length of a single pixel based on the length and the pixel number of the ultrasonic image;
count the tunica intima and externa pixel number in each frame of the tunica intima and externa divided image;
calculate the tunica intima and externa width of each frame of the tunica intima and externa divided image based on the length of the single pixel and the tunica intima and externa pixel number; and
select the greatest tunica intima and externa width as a final tunica intima and externa width to be added into the image parameter set, and transmit a frame of tunica intima and externa divided image with the greatest tunica intima and externa width to a carotid artery stenosis rate calculation unit.

5. The carotid artery ultrasonic examination report generation system based on multi-modal information according to claim 4, wherein the carotid artery stenosis rate calculation unit is configured to:
receive the frame of tunica intima and externa divided image with the greatest tunica intima and externa width, calculate a distance among all tunica intima pixels in a line for each line in an image matrix of the frame of tunica intima and externa divided image, and select the greatest distance as a tunica intima diameter; and
select the smallest diameter and the greatest diameter in all the tunica intima diameters, take a ratio of the smallest diameter to the greatest diameter as a carotid artery stenosis rate, and add the carotid artery stenosis rate to the image parameter set.

6. The carotid artery ultrasonic examination report generation system based on multi-modal information according to claim 3, wherein the plaque area calculation unit is configured to:
calculate a length of a single pixel based on the length and the pixel number of the ultrasonic image;
count the pixel number of the plaque in each frame of the plaque divided image;
calculate a plaque area of each frame of the plaque divided image based on the length of the single pixel and the pixel number of the plaque; and
select the greatest plaque area as a final plaque area to be added to the image parameter set, and transmit the frame of plaque divided image with the greatest plaque area to the plaque volume calculation unit.

7. The carotid artery ultrasonic examination report generation system based on multi-modal information according to claim 6, wherein the plaque volume calculation unit is configured to:
count a frame number of the ultrasonic image with the plaque in the plaque divided image;
receive the frame of plaque divided image with the greatest plaque area, and draw a smallest rectangle capable of completely covering a plaque region with a plaque division boundary as a standard in the frame of image, wherein a length of the rectangle is a plaque major axis diameter, and a width of the rectangle is a plaque minor axis diameter; and
calculate a plaque volume based on the plaque major axis diameter, the plaque minor axis diameter and the frame number of the ultrasonic image with the plaque, and add the plaque volume to the image parameter set.

8. The carotid artery ultrasonic examination report generation system based on multi-modal information according to claim 2, wherein the processor further comprises a user feedback module configured to obtain user feedback information and update the plaque type recognition model, the tunica intima and externa division model or the plaque division model based on the user feedback information.

9. The carotid artery ultrasonic examination report generation system based on multi-modal information according to claim 8, wherein the user feedback information is one or more of no modification, a final conclusion modification result, a tunica intima and externa divided image modification result and a plaque divided image modification result;
the final conclusion modification result is used as feedback to be input into the plaque type recognition module, a modified plaque type and corresponding carotid artery multi-modal information are added into a training set to retrain the plaque type recognition model to update parameters of the plaque type recognition model;
the tunica intima and externa divided image modification result is used as feedback to be input into the tunica intima and externa division unit to update parameters of the tunica intima and externa division model; and
the plaque divided image modification result is used as feedback to be input into the plaque division unit to update parameters of the plaque division model.

10. The carotid artery ultrasonic examination report generation system based on multi-modal information according to claim 2, further comprising a display device connected with the processor and configured to display the tunica intima and externa divided image, the plaque divided image and the carotid artery ultrasonic examination report.
